(19) United States
(12) Patent Application Publication
(10) Pub. No.: US 2012/0172847 A1
(43) Pub. Date: Jul. 5, 2012

(54) VACUUM ASSIST DEVICE TO REMOVE OBJECT FROM TRACHEA

(76) Inventor: Nicholas W. Hooper, Laguna Beach, CA (US)

(21) Appl. No.: 12/982,710

(22) Filed: Dec. 30, 2010

(51) Int. Cl. A61M 1/00

(52) U.S. Cl. 604/540

(57) ABSTRACT

A semi-rigid tube connected to an inflatable ring and a vacuum source is provided. The inflatable ring may be inserted into the trachea air passageway of a person, then inflated to form a seal against the inner surface of the air passageway of the person. A central through hole of the ring is in fluid communication with the tube as well as a lower portion of the air passageway of the person below the ring. The vacuum source applies a vacuum to the tube as well as the central through hole of the ring and the lower portion of the air passageway of the person to remove lodged objects stuck in the air passageway of the person. Once the lodged object is removed from the air passageway, the ring may be deflated and the tube/ring removed from the air passageway of the person so that the person can now breathe normally.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The present invention relates to an apparatus for removing an object lodged within a trachea of a person.

People of all ages choke on various objects. Babies will choke on play things. Adults may choke on food. The basic maneuver to dislodge objects lodged within the air passageway of a person is referred to as the Heimlich maneuver. The person conducting the maneuver must stand behind the victim and wrap his/her arms around the choking victim. The person conducting the maneuver places his/her thumb against the victim's upper abdomen, above the naval but below the breast bone. The hands are clasped together and the person pulls forcefully into the abdomen with a quick upward and inward thrust. The purpose is to push air from the lungs up into the windpipes to dislodge the stuck object. The maneuver is repeated several times until the object is dislodged.

Unfortunately, if the victim is unconscious, then the person conducting the maneuver must be capable of holding the person up while conducting the maneuver properly. If he or she is not capable, then the choking victim is laid on his/her back as the person conducting the maneuver straddles the choking victim. The heel of the hand is placed on the abdomen of the choking victim with the other hand on top of the first hand to perform a quick forceful push. Once again the procedure attempts to push air out of the lungs and through the windpipe to dislodge the foreign object.

Another maneuver is to hold the person's tongue with one hand while reaching in with two (2) fingers to remove the lodged object. The danger is that the lodged object would be pushed further down into the throat.

Unfortunately, the above maneuvers may be ineffective and are also complicated by the fact that the choking victim may be unconscious and/or unable to position his/her body in the upright position.

Accordingly, there is a substantial need in the art for an improved method and/or device for removing clearing an air passageway of a person.

### BRIEF SUMMARY

The present invention specifically addresses the issues identified above, discussed below and those that are known in the art.

An apparatus for removing an object lodged within an air passageway of a person is disclosed. The apparatus may include a semi-rigid tube that can conform to a shape of the air passageway of the person and not collapse when a vacuum is applied to the tube. At the end of the tube, an inflatable ring may be attached. The inflatable ring has a central through hole which is in fluid communication with a lumen of the tube. The tube is also in fluid communication with the vacuum source that applies a vacuum pressure to the tube as well as the lumen of the inflatable ring. During use, the inflatable ring is initially deflated. While deflated, the inflatable ring is inserted through the air passageway of the person choking on an object. Once the ring is positioned within the air passageway of the choking victim, the inflatable ring is inflated to form a seal about an inner surface of the air passageway of the person. Once inflated, the vacuum source applies a vacuum to the tube and the lumen of the inflated ring. The vacuum urges the object stuck in the air passageway of the choking victim through the lumen of the inflated ring and into the tube. Once the lodged object is removed from the air passageway and fed into the lumen of the inflated ring or the tube, the inflatable ring is deflated and removed from the air passageway of the person. The person can now breathe normally.

More particularly, an apparatus for removing an object stuck within a lower portion of an air passageway of a person is disclosed. The apparatus may comprise an inflatable ring, a tube, and a vacuum source. The inflatable ring may be sized and configured to be insertable into the air passageway of the person when the ring is deflated. After insertion, the inflatable ring may form a seal with an interior surface defining the air passageway when the ring is inflated. The ring may have a central hole that is in fluid communication with the lower portion of the air passageway of the person when the ring is inserted into the air passage way and inflated.

The tube may be in fluid communication with the central hole of the ring.

The vacuum source may be attached to and in fluid communication with the tube for providing suction to the lower portion of the air passageway of the person for moving the object from the lower portion of the air passageway, through the central hole and through the tube to remove the object from the lower portion of the air passageway of the person.

The ring may define an inner periphery. The apparatus may further comprise a fitting which defines first and second end portions. The first end is attached to the tube and the second end has an opening which matches the inner periphery of the ring to maximize the size of the object that can be removed from the air passage of the person.

The ring may have an oval shape.

The apparatus may further comprise a mechanical pump in fluid communication with the ring for inflating the ring and a valve disposed between the pump and the ring for maintaining or releasing gas in the ring.

The pump may be a bulb.

The vacuum source may be a syringe, pump or vacuum.

Additionally, a method of removing an object from a lower portion of an air passageway of a person is disclosed. The method may comprise the steps of inserting a deflated ring into the air passageway of the person; inflating the ring; forming a seal between the ring and the interior surface of the air passageway; positioning a central hole of the ring to be in fluid communication with a lower portion of the air passageway of the person; and applying a negative pressure to a tube in fluid communication with the central hole of the ring to dislodge and remove the object located at the lower portion of the air passageway. The applying the vacuum step may comprise the step of actuating a syringe, pump or vacuum to induce the negative pressure to the tube.

The inflating the ring step may comprise the step of actuating a pump in fluid communication with the ring until the desired seal is established.

The method may further comprise the step of closing a valve disposed between the pump and the ring to maintain pressure within the ring.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a perspective view of a vacuum assist device to remove an object from an air passageway of a choking victim;

FIG. 2 is a plan view of the device shown in FIG. 1;

FIG. 3 is an exploded plan view of a plunger of the device shown in FIG. 1; and

FIG. 4 is a cross sectional view of a person with the plunger inserted into the air passageway of the person.

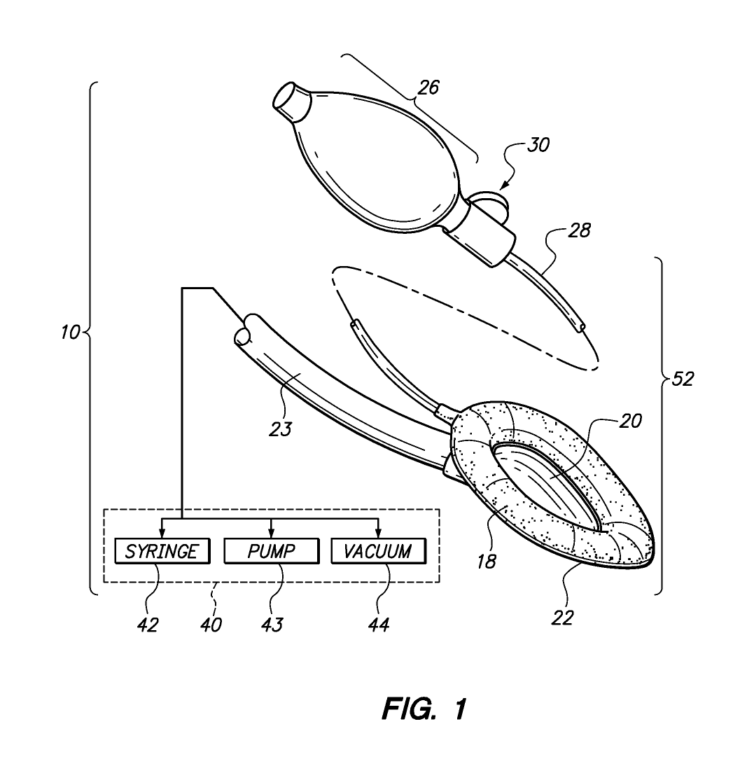

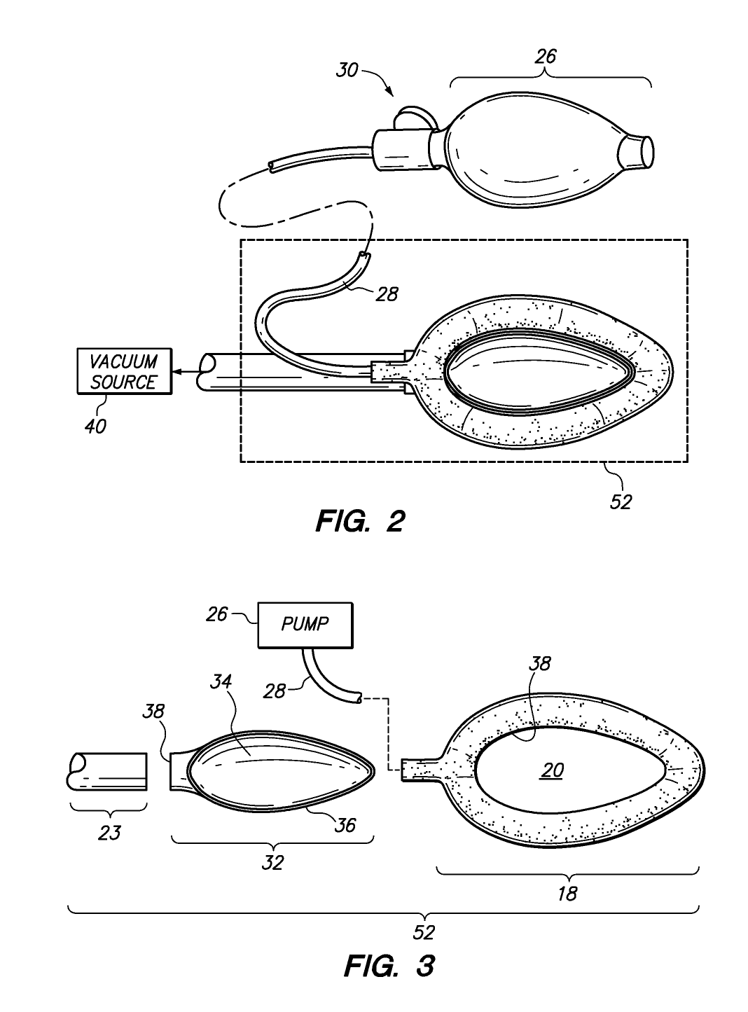

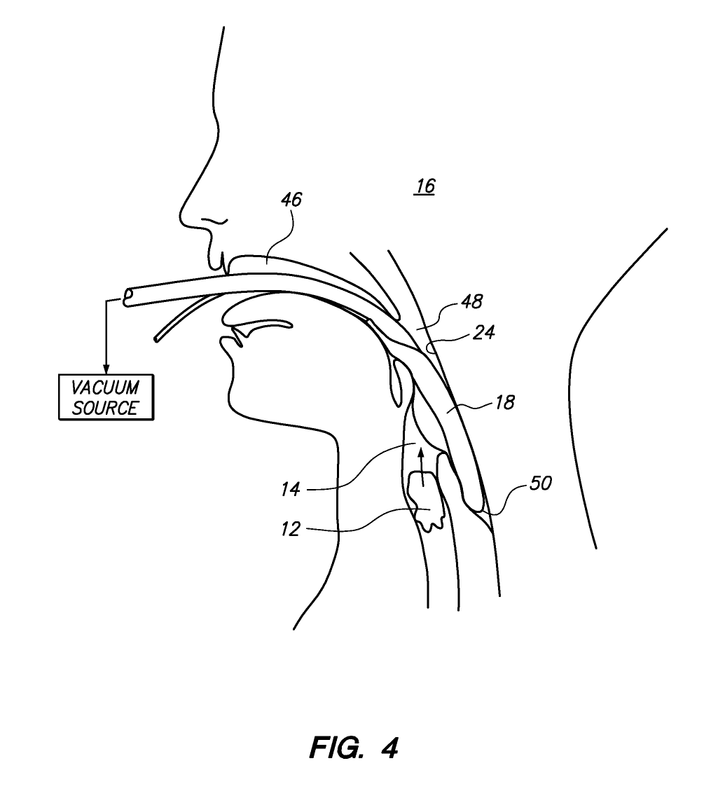

### DETAILED DESCRIPTION

Referring now to the drawings, an apparatus 10 is shown for safely removing a lodged object 12 that is stuck in an air passageway, i.e. trachea, 14 of a person 16. The apparatus 10 may be utilized by a lay person and does not require special training or certification. The apparatus 10 may comprise an inflatable ring 18 that may be inserted into the air passageway 14 of the person 16. The inflatable ring 18 has a central lumen 20 which can be positioned in the person's air passageway so as to be in fluid communication with the air passageway 14 of the person 16 when the apparatus 10 is in use. The lumen 20 is in fluid communication with a tube 23 that extends from the inflatable ring 18 and out of the person's mouth. The inflatable ring 18 can form a seal about an interior wall 24 of the air passageway of the person 16. After the seal is formed, a vacuum may be applied to the tube 22 to remove objects lodged in the air passageway (e.g. trachea) of the person and out of the tube 22. After removal, the inflatable ring 18 is deflated and the apparatus 10 is removed from the air passageway of the person. The apparatus or certain components thereof can be thrown away and a new clean apparatus 10 used the next time. Alternatively, the apparatus can be sterilized for subsequent use.

More particularly, the inflatable ring 18 may be fabricated from a flexible latex, plastic, or rubber material. The inflatable ring 18 may have an oval or doughnut shape which forms a circular ring configuration with a central through hole or lumen. The inflatable ring 18 may be sized such that when deflated, the inflatable ring 20 can be inserted through the air passageway of the person. However, when the inflatable ring 18 is inflated, the outer peripheral portion 22 of the inflatable ring 18 forms a generally air tight seal with the inner wall 24 of the air passageway. When the vacuum is applied, a pressure differential exists between the downstream and upstream sections of the air passageway with respect to the object 12 lodged in the air passageway 14 (e.g., trachea) of the person 16 to force the object in toward the ring 18.

To inflate the ring 18, the ring 18 may be connected to a pump 26. The pump 26 may be squeezed repeatedly to force air or other gas into the ring 18 to inflate the same. The pump 26 may be in fluid communication with the inflatable ring 18 by way of tube 28. Each time that the pump 26 is squeezed, air is introduced into the inflatable ring to inflate the inflatable ring 18. When the inflatable ring is fully inflated so that the inflatable ring 18 forms a seal about the inner wall 24 of the air passageway 14, a valve 30 is closed to prevent leakage or deflation of the inflated ring 18. After the object 12 is dislodged from the air passageway 14 of the person 16, the valve 30 is opened to deflate the inflated ring 18 for removal of the apparatus 10 from the air passageway 14 of person 16.

Referring now to FIG. 3, the inflatable ring 18 can be connected to a fitting 32. The fitting 32 defines a first opening 34. The first opening 34 has a periphery 36 that is sized to be equal or greater than an inner periphery 38 of the inflatable ring 18. This allows the apparatus 10 to remove as large an object 12 as possible through the lumen 20 of the ring 18, fitting 32 and tube 23. Even if the object 12 does not pass through the fitting 32 and into the tube 23, the object 12 could remain lodged or stuck in the lumen 20 of the inflatable ring 18 or in the opening 34 of the fitting 32. When the apparatus 10 is removed from the air passageway of the person 16, the object 12 would be removed at the same time. The fitting 32 has a second opening 38 which generally has a smaller cross sectional opening than the first opening 34. The second opening 38 receives the tube 22 and forms an airtight seal therebetween. The tube as well as the fitting 32 are semi-rigid so that the tube 22 and fitting 32 do not collapse upon application of a vacuum yet are flexible enough to be bent or flexed to conform to the shape of the air passageway of person 16.

A vacuum source 40 may be connected to the proximal end of the tube 22 to provide negative pressure to the lumen 20 of the inflatable ring 18. When the negative pressure is applied, objects lodged within the air passageway are urged toward the lumen 20 of the ring 18 and out of the air passageway. The vacuum source may be a syringe 42, pump 43 or a vacuum 44.

In use, the inflatable ring is inserted through the mouth of the person, down through the throat and past the epiglottis until a distal end 50 of the ring 18 is in the base of the throat lying against the upper end of the normally closed oesophagus. At this time, the ring 18 is inflated to seal the ring 18 to the interior walls 24 of the air passageway 14 or, more particularly, the larynx.

After the ring 18 is sealed to the interior walls 24 of the air passageway 14, a vacuum is applied to the tube 23. Objects lodged in the air passageway 14 below the ring 18 are urged upward toward the lumen 20 of the ring 18. Preferably, the object 12 passes through the lumen, through the fitting 32 and out of the tube 23 to clear the air passageway 14 of the choking person. When the apparatus 10 is removed from the air passageway 14 of the choking person, the air passageway 14 is now clear and the person can now breathe normally.

If the object 12 is too large to fit through the tube 23, then the object can still be removed from the air passageway 14 of the person 16. When the vacuum is applied to the tube 23, the object moves upward and into the lumen 20 of the ring 18. However, since the object 12 is too large, the object can now be wedged in or secured to the lumen 20 of the ring 18. For example, the vacuum can be maintained while the apparatus 10 is removed from the air passageway 14 of the person. Alternatively, the object can be wedge in the fitting 32. During removal of the apparatus 10, it is contemplated that the ring 18 may be deflated by opening the valve 30. Also, the vacuum may be optionally maintained so that the object remains lodged within the lumen 20 of the ring 18 or against the lumen of the tube 23.

The apparatus may be provided as a kit. The complete kit may include the vacuum source 40, the tube 28, tube 23, fitting 32, the inflatable ring 18 which may be referred to as a plunger 52 and the pump 26. The plunger 52 is removably attached to the vacuum source 40 and the pump 26. In particular, the tube 23 may have a mating fitting with the vacuum source 40. Likewise, the tube 28 may have a fitting that can be removably attachable to the pump 26. Initially, the operator is provided with these three (3) components, namely, vacuum source 40, plunger 52 and pump 26. Prior to use, these components may be assembled and stored in a sterile pouch so that when needed, the apparatus 10 may be quickly and immediately deployed since time is of the essence. During use, the plunger 52 is inserted into the air passageway of the choking person. The external as well as internal surfaces of the tube 23 and the ring 18 are contaminated by the person's saliva, germs, viruses, food particle matter, etc. while removing and dislodging the object stuck in the person's air passageway. After the procedure is performed, the operator may remove the pump 26 from the tube 28 and remove the vacuum source 40 from the tube 23. The plunger 52, pump 26 and/or tube 28 may then be disposed or thrown away. Alternatively, the plunger 52, pump 26 and/or tube 28 may be sterilized and repackaged for subsequent use.

The plunger 52 (i.e., the tube 28, tube 23, fitting 32, the inflatable ring 18) can be fabricated in different sizes so that the apparatus can be deployed to fit a child, teenager, young adult or adult. By way of example and not limitation, the length and diameter of tube 23 and the ring 18 may be provided in different sizes in a kit to fit a small child, teenager, young adult and a grown adult. Accordingly, the kit may include one vacuum kit and one pump 26, one plunger 52 sized for a small child, one plunger 52 sized for a teenager, one plunger 52 sized for a young adult and one plunger 52 for a grown adult. When needed, any one of the plungers 52 that will fit the person choking may be quickly attached to the vacuum source 40 and the pump 26.

The apparatus 10 disclosed herein may be employed by a layperson, emergency medical technician, etc. to provide basic life support while a choking victim or injured individual is being taken to a hospital or a doctor.

The above description is given by way of example, and not limitation. Given the above disclosure, one skilled in the art could devise variations that are within the scope and spirit of the invention disclosed herein, including various ways of pumping the ring 18 and applying a vacuum. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. An apparatus for removing an object stuck within a lower portion of an air passageway of a person, the apparatus comprising:
an inflatable ring which is sized and configured to be insertable into the air passageway when the ring is deflated and to form a seal with an interior surface defining the air passageway when the ring is inflated wherein the ring has a central hole that is in fluid communication with the lower portion of the air passageway of the person when the ring is inserted into the air passage way and inflated;
a tube in fluid communication with the central hole of the ring; and
a vacuum source attached to and in fluid communication with the tube for providing suction to the lower portion of the air passageway of the person for moving the object from the lower portion of the air passageway, through the central hole and through the tube to remove the object from the lower portion of the air passageway of the person.

2. The apparatus of claim 1 wherein the ring defines an inner periphery, and the apparatus further comprises a fitting which defines first and second end portions, the first end is attached to the tube and the second end has an opening which matches the inner periphery of the ring to maximize the size of the object that can be removed from the air passage of the person.

3. The apparatus of claim 1 wherein the ring has an oval shape.

4. The apparatus of claim 1 further comprising a mechanical pump in fluid communication with the ring for inflating the ring and a valve disposed between the pump and the ring for maintaining or releasing gas in the ring.

5. The apparatus of claim 4 wherein the pump is a bulb.

6. The apparatus of claim 1 wherein the vacuum source is a syringe, pump or vacuum.

7. A method of removing an object from a lower portion of an air passageway of a person, the method comprising the steps of:
inserting a deflated ring into the air passageway of the person;
inflating the ring;
forming a seal between the ring and the interior surface of the air passageway;
positioning a central hole of the ring to be in fluid communication with a lower portion of the air passageway of the person;
applying a negative pressure to a tube in fluid communication with the central hole of the ring to dislodge and remove the object located at the lower portion of the air passageway.

8. The method of claim 7 wherein the inflating the ring step comprises the step of actuating a pump in fluid communication with the ring until the desired seal is established.

9. The method of claim 8 further comprising the step of closing a valve disposed between the pump and the ring to maintain pressure within the ring.

10. The method of claim 7 wherein the applying the vacuum step comprises the step of actuating a syringe, pump or vacuum to induce the negative pressure to the tube.
